(19) United States
(12) Patent Application Publication
(10) Pub. No.: US 2007/0065805 A1
(43) Pub. Date: Mar. 22, 2007

(54) Cell migration assay

(75) Inventors: Liming Yu, Clifton Park, NY (US); Lihong Zhao, Mountainside, NJ (US); Anton Beletskii, Niskayuna, NY (US); Padma Channavajhala, Niskayuna, NY (US)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE Healthcare Bio-Sciences Corp.

(21) Appl. No.: 11/519,643

(22) Filed: Sep. 12, 2006

Related U.S. Application Data
(60) Provisional application No. 60/718,057, filed on Sep. 16, 2005.
(60) Provisional application No. 60/747,430, filed on May 17, 2006.

(51) Int. Cl. C12Q 1/00; C12N 5/02

(52) U.S. Cl. 435/4; 435/405

(57) ABSTRACT

The present invention provides compositions and methods for preparation of a three-dimensional transendothelial cell migration (TEM) assay. These compositions and methods are uniquely suited for the high throughput TEM assay, and for the analysis and identification of TEM mediators which inhibit or stimulate this process. The composition for detecting migration of cells comprises a solid layer comprising collagen gel; a first cellular layer in contact with the solid layer and comprising a first cell type; and a second cell type seeded on top of the first cellular layer. Optionally, gelatin is included in the collagen gel. A 96 well plate format is disclosed, the combination with a high throughput cellular scanner enables high throughput TEM assay.

### CROSS-REFERENCE TO RELATED APPLICATIONS

This application claims priority to U.S. provisional patent application No. 60/718,057 filed Sep. 16, 2005 and to U.S. provisional patent application No. 60/747,430 filed May 17, 2006; the disclosures of which are incorporated herein by reference in their entireties.

### FIELD OF THE INVENTION

The present invention relates generally to methods of a diapedesis assay. More specifically, it relates to compositions for a transendothelial migration assay, methods for preparing and methods for using these compositions.

### BACKGROUND OF THE INVENTION

Migration of cells through vascular endothelium is a key event in the pathophysiology of conditions such as inflammation, atherosclerosis and tumor metastasis. Methods have been developed over the years for the measurement of cell migration in vitro. The most commonly used methods involve an artificial barrier (membrane), and usually require manual counting of migrated cells. Existing devices on the market for cell migration assay are: classic Boyden chamber, cell culture insert (a modified version of Boyden chamber), FluoroBlock™ BD Biosciences), and Cell Motility HitKit™ (Cellomics). The major limitations associated with these devices are low throughput, manual cell counting, usage of biologically irrelevant materials for cells to cross, and difficulty in analyzing the out put results.

Recently, multilayered set-ups have been proposed, in an effort to mimic the in vivo environment of the migrating cells (See International Application Publication number WO 2003/027256 and WO 2004/046337). However, the systems are complex to prepare, and are not suited for high throughput screening.

There remains a need for an improved, simple to use Transendothelial Cell Migration (TEM) assay system, especially for high throughput screening in the drug discovery industry.

### SUMMARY OF THE INVENTION

The objectives of the invention are to provide compositions and methods for transendothelial cell migration assay. These compositions and methods are uniquely suited for the high throughput TEM assay, and for the analysis of TEM mediators which inhibit or stimulate this process.

One aspect of the invention provides a composition of matter for detecting migration of cells, which composition comprises a solid layer comprising collagen gel; a first cellular layer in contact with said solid layer and comprising a first cell type; and a second cell type seeded on top of the first cellular layer. Optionally, gelatine is included in the solid, collagen gel layer. One specific embodiment of this aspect provides the composition in a 96 well plate format, with a confluent first cellular layer of human umbilical vein endothelial cells (HUVEC), and neutrophil or peripheral blood mononuclear cells (PBMC) as the second cell type. Variations of this embodiment are provided in the detailed descriptions and the claims that follow.

Another aspect of the invention provides a method for preparing the composition of matter for the detection of cell migration, comprising the steps of: depositing and solidifying collagen gel in a vessel to form a solid layer comprising collagen gel; placing cells of a first cell type on the solid layer and incubating the first cell type to form a confluent cellular layer in contact with the solid layer; and seeding cells of a second cell type on top of the first cellular layer. Detailed embodiments are provided that enables the preparation of the composition of matter, including one that is in the 96 well plate format, which is ideal for high throughput analysis of cell migration, including TEM. Optionally, a gelatin solution is mixed with the collagen gel prior to the formation of a solid layer.

Yet another aspect of the invention provides a method of detecting cell migration, including TEM, comprising the steps of: incubating the composition of matter; and detecting migrated cells at a first position of the solid layer of the composition. It is provided that certain embodiments of the method adopt a composition in the 96 well plate format, and is suited for automated, high throughput analysis of cell migration by an automated cell analyzer.

Still another aspect of the invention provides a method for identifying a mediator of cell migration comprising: incorporating a candidate mediator of cell migration into the composition of matter; incubating the composition; and measuring cell migration in the presence of the candidate mediator, wherein a difference in response relative to a composition lacking the candidate mediator identifies a mediator of cell migration. High throughput implementations of this method provides a platform for the rapid testing of large number of cell migration/TEM mediators, and is a key enabler for the pharmaceutical industry.

Other aspects and advantages of the present invention will appear from the detailed description that follows.

### BREIF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows the 3-dimensional Transendothelial Cell Migration (TEM) assay set-up according to the embodiments of the present invention. On the left side is a schematic description of the system from the side view. On the right shows a picture of the endothelial cell (EC) monolayer from the top view.

FIG. 2 is a diagram showing the effect of collagen gel quality on neutrophil TEM.

FIG. 3 is a diagram showing the effect of collagen gel quality on peripheral blood mononuclear cells (PBMC) TEM.

FIG. 4 shows the effect of collagen gel volume on neutrophil TEM.

FIG. 5 shows the effect of collagen gel volume on PBMC TEM.

FIG. 6 shows the effect of starting cell density on neutrophil TEM.

FIG. 7 shows a time course for neutrophil TEM. The migrated cells were quantified at Z: 120 μm above the plate bottom at time points of 0.5, 1, 1.5 and 2 hours.

FIG. 8 shows a time course for PBMC TEM. The migrated cells were quantified at Z: 120 μm above the plate bottom at time points of 2, 4, 6, and 8 hours.

FIG. 9 shows an increase of neutrophil TEM when the gel layer is pre-soaked with IL-8.

FIG. 10 shows that 1, 10-phenathronoline, an MMP-9 inhibitor, inhibits neutrophil TEM.

FIG. 11 is a 3-dimensional image reconstitution of a stack of 21-Z slices through the gel layer.

FIG. 12 is a large scale study of neutrophil TEM with positive controls (IL-1 β stimulated) and negative controls (without IL-1 β stimulation).

### DETAILED DESCRIPTION OF THE PREFERRED EMBODIMENTS

We provide compositions and methods for cell migration assays, including transendothelial cell migration (TEM) and Diapedesis assays. The 3-dimensional assay system is designed to more closely represent the in-vivo situation. The assays have been provided in 96-well format which enables automation for high throughput screening and thus meet the needs for cell-based functional assays in the drug industry. Our results indicate that the assays are suitable for the study of patho-physiologically conditions, such as inflammation, atherosclerosis and tumor metastasis. They are ideally suited for high throughput screening assays. The compositions and methods also provide synergies between improved assay biology and automation feature of cellular analyzers (e.g. IN Cell Analyzer 3000) for quantitative analysis. They provide unique tools for the study of mediators, e.g. cytokine or drugs, that inhibit or stimulate this process.

As used herein, the term “transendothelial migration” (TEM) refers to the movement of migrating cells from the apical surface to the basal lamina of endothelial cells and beyond in response to chemotactic factors (when such factors are present at a higher concentration at the basal lamina than at the apical surface of the endothelial cells). Leukocytes migrate between junctions formed in the endothelium between individual endothelial cells. Generally, TEM occurs when the endothelial cells are activated, e.g., with TNF, IL-1, or other pro-inflammatory mediators. TEM can also occur endogenously, and will occur at a lower, less robust level across endothelial cells as a consequence of leukocyte adhesion even in the absence of direct activation of the endothelial cells. Thus, TEM occurs in vivo at inflammatory foci; and in vitro, across cultured endothelial cells preferably after activation of the endothelial cells and/or creating a chemotactic gradient.

As used herein, the term “Diapedesis” means the movement of leukocytes across the endothelial lining of blood vessels to interstitial fluid (IF). The process is driven by chemotactic factors. Diapedesis usually happens when an area is injured or damaged and an inflammation response is needed.

The Composition

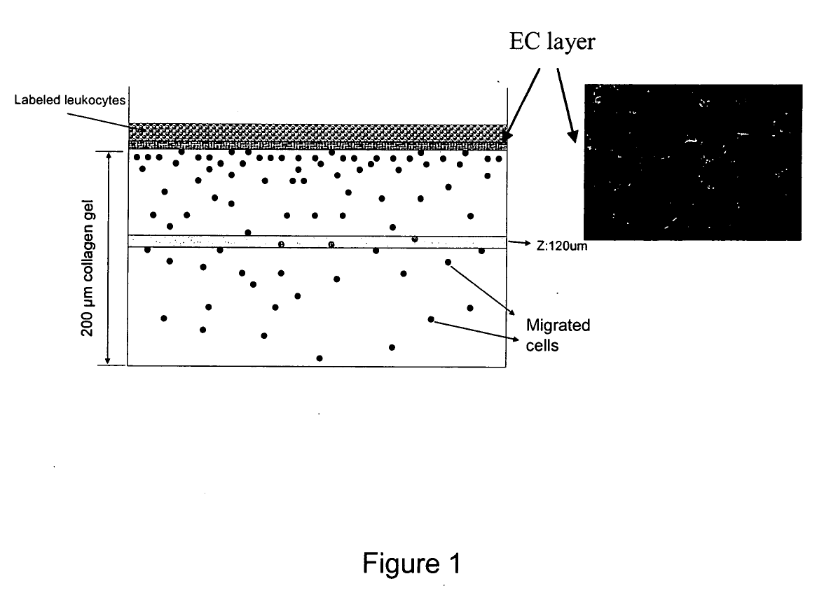

FIG. 1 provides a 3-dimensional Transendothelial Cell Migration (TEM) assay model according to the embodiments of the present invention. On the left side is a schematic description of the system. The top green dots represent fluorescence labelled leukocytes. The brown band represents a confluent endothelial cell. The clear area represents the solidified collagen gel in the thickness of about 200 μm. The scattered green dots below EC layer represent the migrated cells. Note that optionally, gelatine is included in the solidified collagen gel. Note that the thickness of the collagen gel layer is dependent upon the focus plain of the Imager microscope. In most instances, a collagen layer of about 50-500 μm provides a suitable thickness for the TEM assay. On the right shows a photograph of the endothelial cell (EC) monolayer. In the photograph, the nuclei are stained by Hoechst (blue). F-Actin is stained by Alexa Fluor™ 488 conjugated phalloidin (green). The red staining reveals a protein, VE-Cadherin, which is expressed at cell boundaries when a tight-junction is formed.

Our system provides advantages over the prior art systems in that it is simple yet robust. It is inducible and mimics in vivo diapedesis employing human umbilical vein endothelial cells and human leukocytes. We established that neutrophil TEM plateaus within 2 hrs and was shown to be specifically inhibited by MMP-9 inhibitors. We also conclude that the system could be used for chemotaxis study with the addition of chemoattractant molecules in the collagen gel.

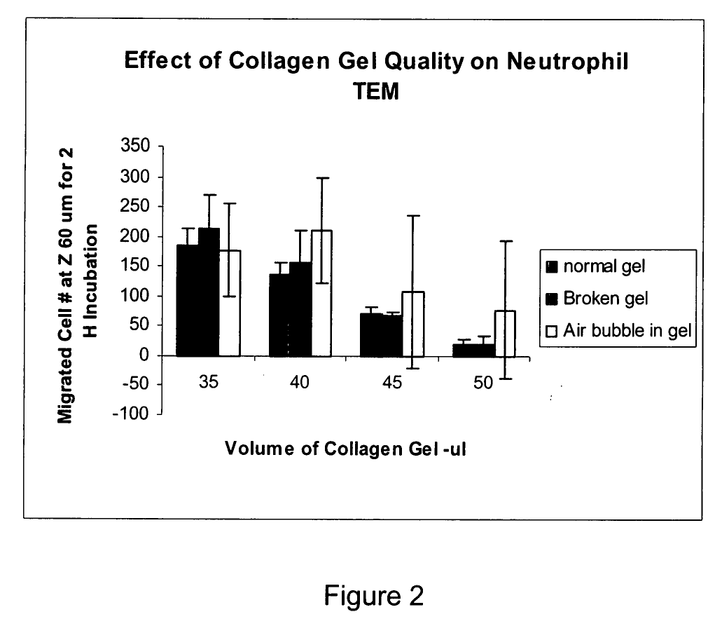

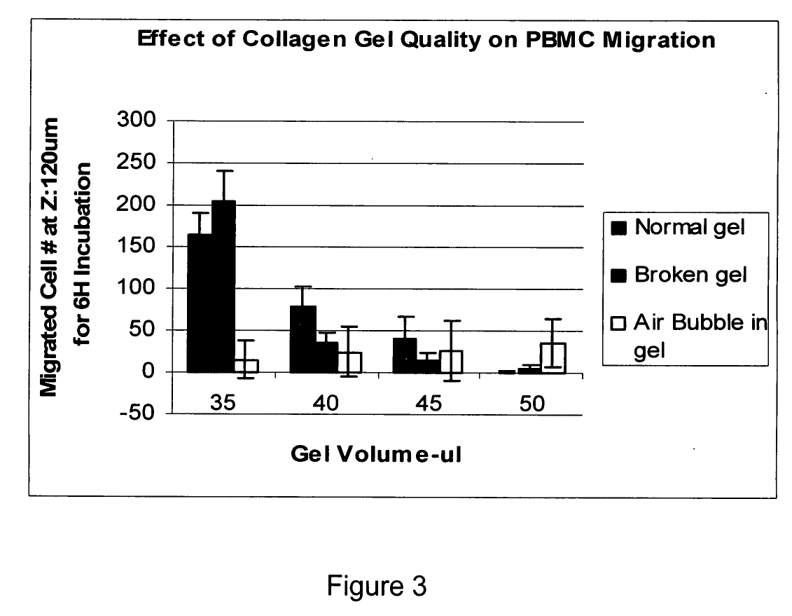

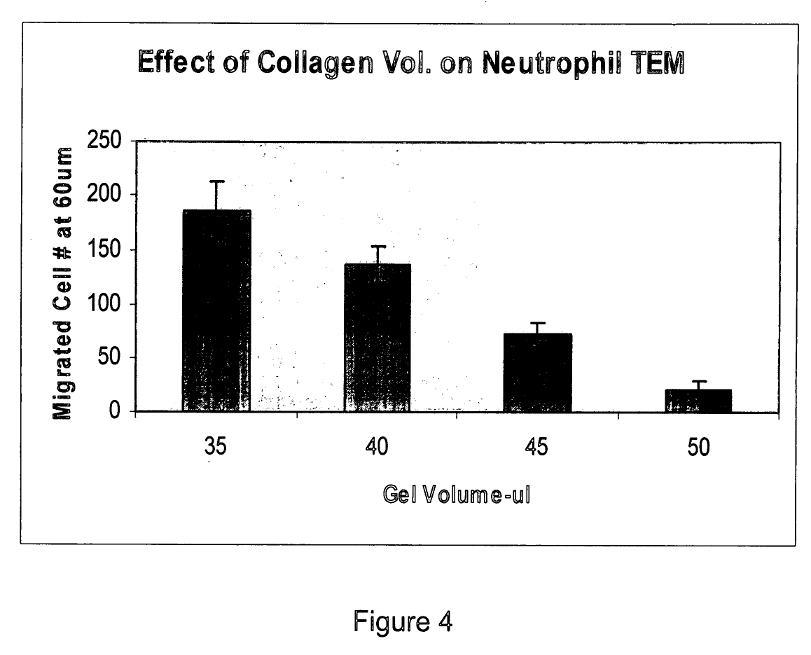

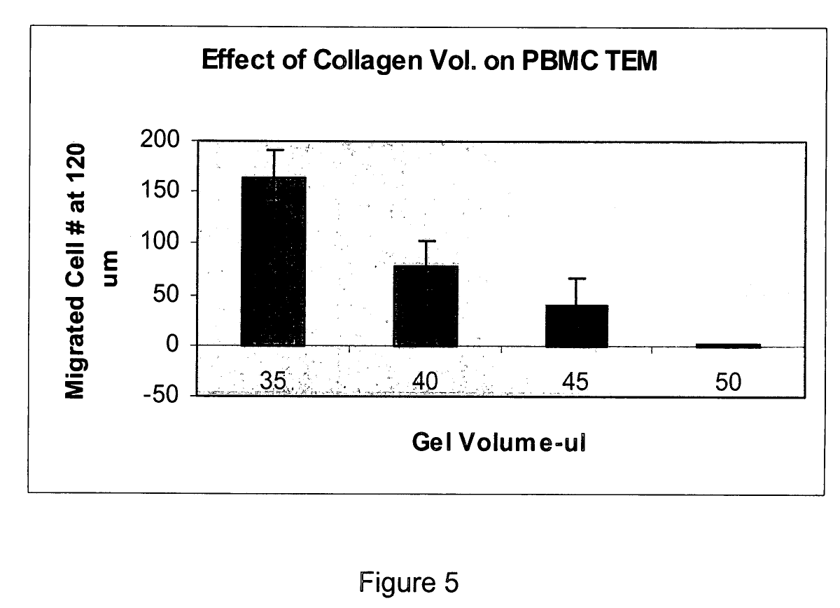

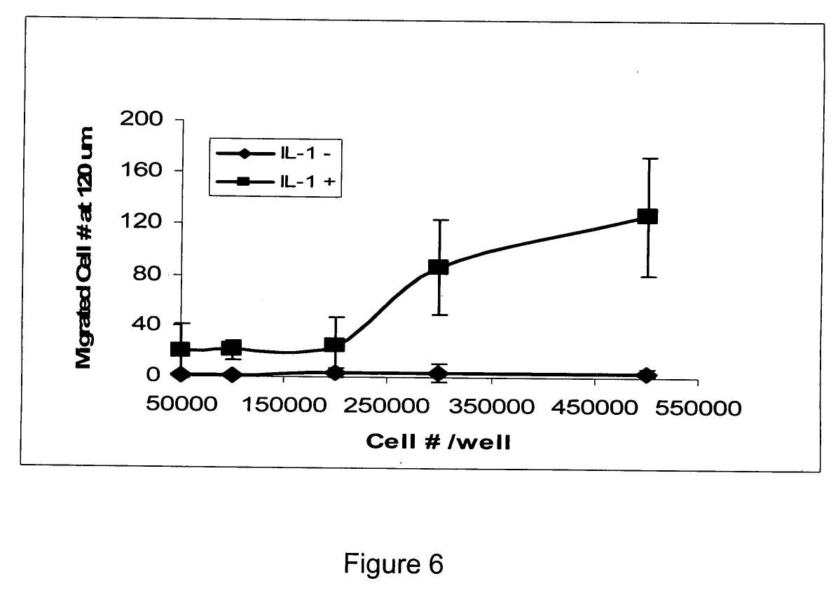

It is noted that the 3-D TEM model diagrams in FIG. 1 represents the set up in a single vessel. We describe in detail, in the Examples section, the development of a 96-well plate format. We established the effects of collagen quality on neutrophil TEM (FIG. 2) and PBMC TEM (FIG. 3). We demonstrated that under normal gel loading conditions and with a quick spin, we can reliably produce collagen gel layers of sufficient quality. We also established a working volume of collagen gel for each well within a standard 96 well plate (FIG. 4 and 5). We also tested for optimal migrating cell density, and concluded that between 300,000-500,000 cells/well gives satisfactory assay results (FIG. 6). We note that the addition of gelatin in the collagen gel layer does not affect the performance of the system. The use of gelatin enables prolonged storage of the solidified collagen gel at room temperature.

The TEM model in the 96-well format offers several advantages. For one, it is a more compact system that allows assay to be performed in a single well of a 96-well plate. The use of an automated cellular analyser and with proper image analysis software enables high throughput drug screening assay. It also allows a quantitative measurement of cell movement in spatial and temporal fashion, and in three dimensions (Z-stacking feature of confocal microscopes). The three-layer set up of the assay system also avoids the use of biologically irrelevant materials such as plastic porous membrane.

Preparation of the Composition

We provide detailed materials and methods for the preparation of the assay system in the Examples section. Briefly, collagen gel is deposited in a vessel and is solidified to form a solid layer. Or alternatively, one can use a synthetic matrix gel which supports the 3D endothelial growth and cell migration. Optionally, a gelatin solution is added to the collagen gel prior to solidification of the collagen layer. Then a first cell type (endothelial cell) is placed on the solid layer and incubated to form a confluent cellular layer in contact with the solid layer. The migrating cells are then prepared and seeded on top of the confluent layer of the first cell type. Typically, the first cell type is an endothelial cell, such as a HUVEC. Other primary endothelial cells, such as HCAEC (coronary artery endothelial cells), HMVEC (lung microvascular endothelial cells), or endothelial cell lines such SK-HEP-1 (ATCC HTB-52), can also be used. We tested primary neutrophil and PBMC as migrating cells, although any migrating cell type could be used and or tested in the system, examples like neutrophil cell line HL-60 (ATCC CCL-240), lymphocytes, tumor cell lines such as HT-1080 (ATCC CCL-121), and spermatozoa.

For the convenience of detection, the migrating cells could be labelled before they are seeded and analyzed. A wide range of dyes commonly used for labelling cells can be used in this model as well, such as Hoechst, Calcein, fluorescein dextran, and Texas Red dextran. As an example, we labelled cells with CellTracker™ Green (Invitrogen) in our study.

Alternatively, a fluorogenic compound can be mixed within the collagen gel during the preparation of the solid collagen gel layer. When cells migrate into the gel, they are exposed to the fluorogenic material. The interaction between cells and the fluorogenic material, such as protease digestion, internalization, or other biochemical reactions, results in fluorescent signal. The signal is then captured by fluorescent microscope and quantitative measurement is performed. Here the migrating cells do not need to be labelled before the assay. This makes the assay easier to perform, more robust, and more suitable for high throughput applications. Because the migrating cells do not possess a label before transendothelial migration, only cells migrated across the endothelial cell layer contain fluorescent signal. Cells that never migrated will not show any signal at all. This eliminates the background from un-migrated, pre-labelled cells, thus increasing assay accuracy and sensitivity.

Method of Using the Composition

The cell migration assay systems we developed can be used for studying cell migration, as well as screening for mediator or drugs that promote or inhibit cell migration. When used to screen mediator of cell migration, the method includes the following steps: (a) incorporate a candidate mediator of cell migration into the composition, or pre-treat the migrating cell with the candidate mediator; (b) incubate the composition, including the seeded migrating cells; (c) measure cell migration in the presence of the candidate mediator; and (d) compare the measured result with that of the same type of cells in the absence of the candidate mediator, a difference in measured migration results identifies a mediator of cell migration.

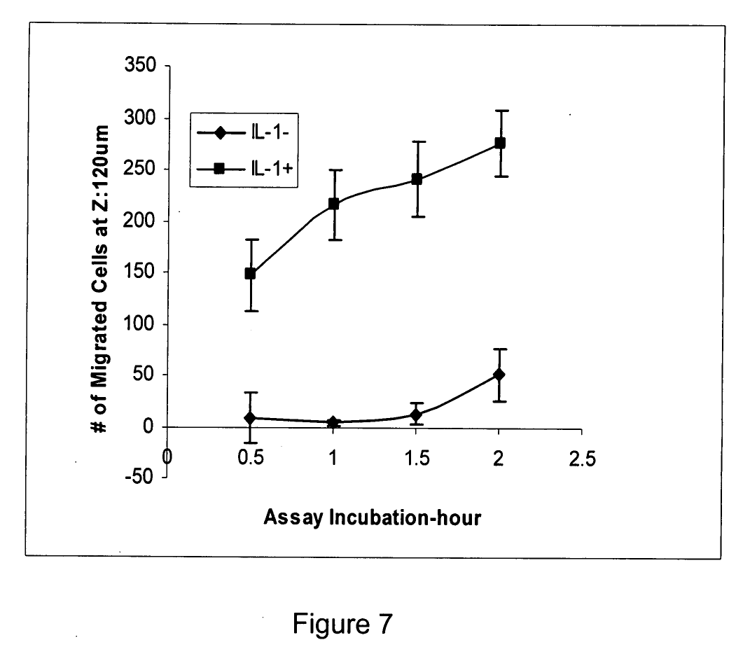

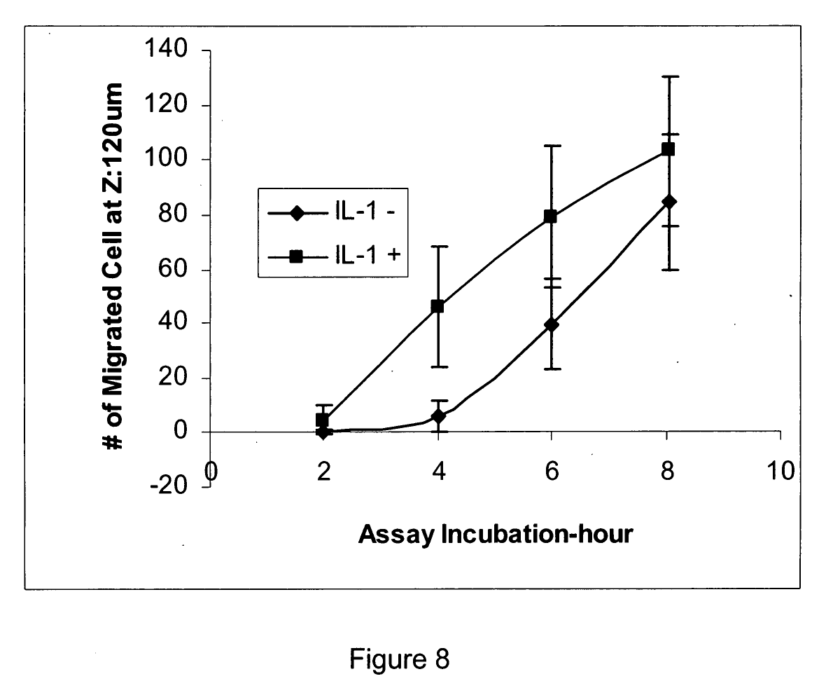

We successfully tested the capability of the system in screening for molecules that stimulate or inhibit cell migration. Interleukin-1-beta (IL-1β) is an endogenous cell migration mediator for both neutrophil and PBMC. IL-1β, clearly stimulates endothelial cells to express cell adhesion molecules which further potentiate transendothelial migration of both cell types. In the presence of IL-1 β, neutrophil TEM happens relatively quickly and reaches a significant signal to noise ratio (S/N) in about 0.5-2 hours (IL-1β stimulated vs. non-stimulated, FIG. 7). We also noticed that the neutrophil TEM S/N (with/without IL-1 β stimulation) reached a plateau with an over-night incubation. PBMC TEM happens relatively slower, requiring an incubation time of 6-8 hours in general (FIG. 8).

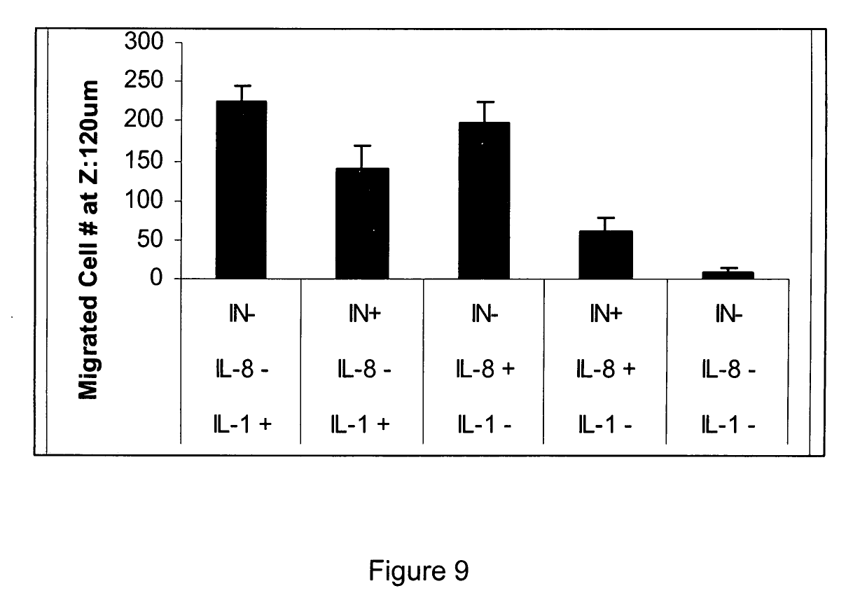

While IL-1 β was added into the culture medium for HUVEC culturing and incubated overnight, we also tested an alternative way of introducing the chemoattractants. Interleukin-8 (IL-8) is a known strong neutrophil attractor. To demonstrate IL-8's effect on neutrophil TEM, we pre-soaked the collagen gel with culture medium containing IL-8 for 4 hours, prior to the seeding of the HUVEC layer. Our results indicate that the soaking of IL-8 generates a TEM effect similar to that of IL-1 β activation of HUVEC (FIG. 9).

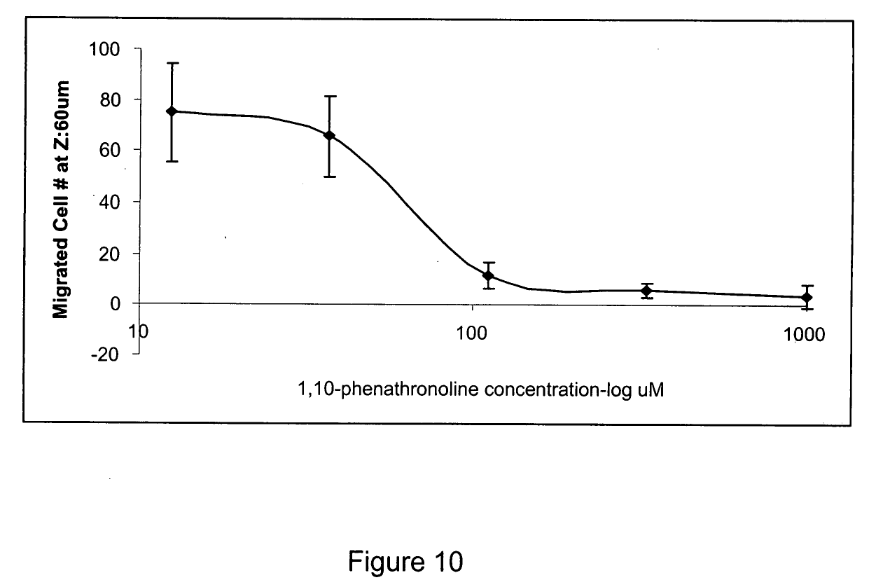

We also tested inhibitors and show that the system could identify TEM inhibitors as well. 1, 10-phenathronoline is known to inhibit MMP-9 (matrix metalloproteinase-9). Neutrophil was pre-treated with 1, 10-phenathronoline. The inhibitor was continuously present throughout the TEM assay. We demonstrated inhibition of neutrophil TEM in FIG. 9 and 10 (For FIG. 9, compare IN−and IN+). Another MMP-9 inhibitor, doxycyclin was also tested and showed to inhibit TEM. It is known that proteases, such as MMP-9, released by a migrating cell will facilitate cell migration through tissue. Inhibition of the protease results in the down regulation of cell migration.

A High Throughput 3-dimensional Cell Migration Assay System

The composition described above has been successfully implemented in a 96-well plate platform. 96-well plates with transparent bottoms are used for the assay, one such example is the ViewPlate™ by PerkinElmer. Analysis of cell migration is performed with an automated cellular analyzer, such as the In Cell Analyze™ (GE Healthcare). As an example, confocal images at a certain Z-plate are generated for a predefined field of view. These images are then processed by automated analysis and quantitation software. The implementation of the assay system in the 96-well format, in combination with the automatic imaging and data analysis, provides a high throughput, cell migration system. This system can be used for the large scale discovery and evaluation of mediators for cell migration, including TEM.

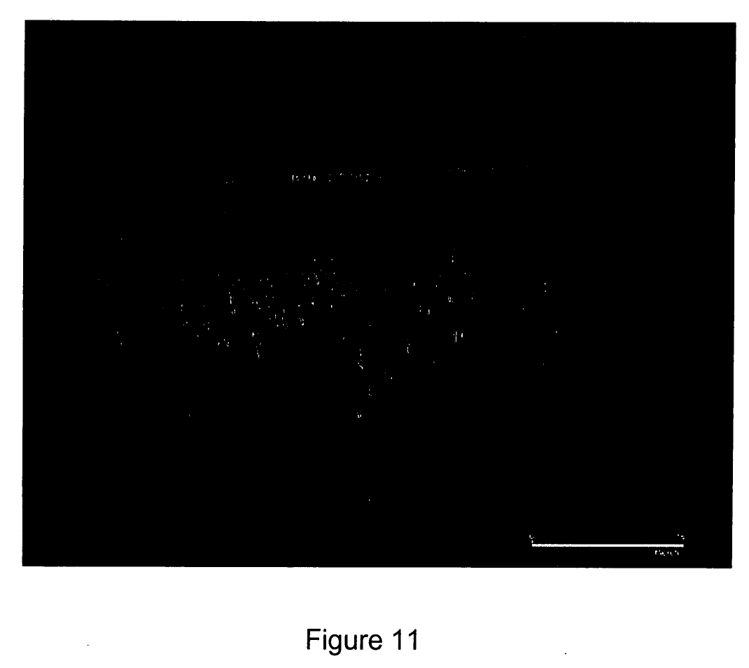

In addition to single Z plane image acquisition, the system can also provide a 3-D cell image of cell migration. Because image acquisition is performed without disturbing the assay system, it can also provide temporal data series. FIG. 11 provided a 3-dimensional image of leukocyte TEM in a field view of a well from a 96-well plate. This image is reconstituted from a stack of 21Z-plate image slices through the collagen gel layer, at 10 μm per section. This 3-D image demonstrates leukocyte TEM in the gel layer, migrating downwards to where the higher gradient chemoattractant(s) were accumulated in the gel

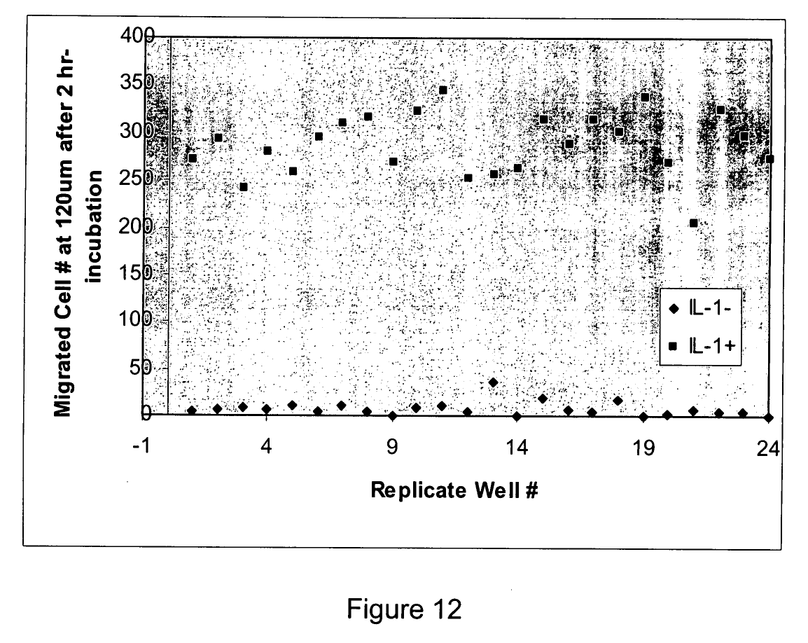

The reliability of the system was tested by a scatter plot of neutrophil TEM, presented as number of migrated cells at Z: 120 μm, with or without HUVEC activation (FIG. 12). We see a tight-scatter distribution. This indicates that variation within the treatments is very small, and the difference between two treatments is significant and distinguishable, which qualifies the assay for high throughput purpose. The same assay format should also be applicable in a 384-well format when needed.

### EXAMPLES

The present examples are provided for illustrative purposes only, and should not be construed as limiting the scope of the present invention as defined by the appended claims. All references given below and elsewhere in the present 5 specification are hereby included herein by reference.

Materials and Methods Table 1 contains a list of essential materials used in the following assays, as well as information about the manufacturers and corresponding catalogue numbers.

Additional materials are described in the methods that follow.

TABLE 1
Materials used in the experimental sections.

| Material | Supplier | Catalogue # |
|---|---|---|
| HUVEC | CAMBREX | CC2915 |
| EGM-2 | CAMBREX | CC3162 |
| Fibronectin | BD Biosciences | 354008 |
| VITROGEN-100, Collagen type I | COHESION | FXP-019 |
| CellTracker Green | Invitrogen | C2925 |
| Alexa Fluor ™ 488 | Invitrogen | A-22284 |
| conjugated phalloidin | | |
| Anti-human PECAM monoclonal | Pierce | MA 3100 |
| Human serum albumin | Sigma | A 1653 |
| RPMI medium | Invitrogen | 72400-047 |
| Cadherin-5, Mouse anti-human | BD Biosciences | 555661 |
| monoclonal antibody | | |
| Mouse IgG | Sigma | M 9144 |
| ViewPlate ™ 96-well plate | PerkinElmer | 6005182 |
| Gelatin | Sigma | G9391 |
| Black adhesive plate seal | Perkin Elmer | 6005189 |

The following subtitles describe the protocols used for the preparation and performance of the assay systems.

Preparation of Collagen Gel Layer

Collagen 1 was prepared following manufacturer's suggestion. Briefly, 8 ml of collagen was mixed with 1 ml of 10 XPBS and 1ml NaOH (0.1 N), using pre-chilled pipette and reagents kept at 4° C. Optionally, the pH of the mixture was adjusted to pH 7.5 by the addition of 0.12N HCI.

A 96-well plate (ViewPlate™, PerkinElmer Life and Analytical Sciences) was set on ice and 40 μl of gel (2.5 mg/ml) was dispensed into each well using stepper repeat pipette (500 or 1000 tip). The plate was spun at 1,500 rpm for 2 min at 4° C. The gel was solidified at 37° C. in a CO2-free incubator to establish a thick layer (200 μm) of collagen gel onto a well of 96-well plate. The following TEM assays were performed using collagen gel prepared in this manner.

Alternatively, a gelatine solution was added to the collagen gel mixture prior to dispensing into wells of a 96 well plate. 5% gelatin solution was prepared by adding 5 grams of the powder to tissue grade water and heating until it dissolved completely. The pH was adjusted to 7.2 with 10 N NaOH, and the solution was sterilized by autoclaving at 121° C. for 30 min. Aliquots of 1 ml volumes were stored at 4° C. 125 μl of 5% gelatin solution was added to every 1 ml of collagen mixture. The collagen/gelatine mixture was dispensed and solidified similar to the collagen gel mixture. The plate with solidified gel can be used right away for TEM assay described below. Alternatively, the plate can be sealed with a plate seal and kept in a humidity environment at room temperature for later use.

Culture of HUVEC to Form a Confluent Monolayer on the Gel

The layer of collagen gel in each well was coated with 200 μl of 1 μg/ml human fibronectin (BD Biosciences) in serum-free EGM-2 medium for 1 hour at room temperature. After removal of the fibronectin containing-medium, HUVEC cells (CAMBREX) were seeded onto the gel and cultured in the EGM-2 medium for 3 days, at 37° C., and at a concentration of 40,000 cells/well. The cells were chosen from early passage (3^{rd} to 4^{th}), 70-80% confluent HUVEC cell cultures. The day before the assay, the HUVEC culture medium was replaced with either fresh EGM-2 medium alone, or the fresh EGM-2 medium containing 10 ng/ml IL-1 β (or TNF-α, or other chemoattractants). The mixture was incubated overnight to stimulate TEM.

Alternatively, to demonstrate that IL-8 is a primary chemoattractant to neutrophil TEM, the collagen gel may be pre-soaked with culture medium containing IL-8 at 200 ng/ml for 4 hours, prior to seeding of the migrating cells.

Isolation and Labelling of Leukocytes from Blood Sample

Neutrophil or peripheral blood mononuclear cells (PBMC) were freshly isolated from blood Buffy coat. Briefly, RBC were removed using dextran sedimentation. Then PBMC were isolated by Ficoll-Hypaque centrifugation. Neutrophils were purified by hypotonic lysis of remaining RBC in the pellet of Ficoll-Hypaque centrifugation. The cells were labelled with CellTracker™ Green, by incubation in 0.5 to 1 μm dye in RPMI for 45 min at 37° C. The dye containing RPMI was removed and the cells washed once with serum-free RPMI medium. The cells were re-suspended in RPMI containing 0.2% HAS (assay medium) at 2.5×10⁶ cells/ml.

Performing and Measuring the TEM Assay

On the assay day, the culture medium for the HUVEC cell culture was removed and the HUVEC monolayer washed 2 times with PBS and once with assay medium (0.2% of HAS in RPMI). 500,000 (200 μl) neutrophil or PBMC, which were CellTracker™ Green labelled, were placed on top of the HUVEC monolayer in each well. The assay was incubated further at 37° C. The length of time for the incubation is primary cell type dependent. For neutrophil, incubation time is within 2 hours and for PBMC, from 6 to 10 hours may be required.

After the required incubation time, images of neutrophil/PBMC cells which had migrated below the HUVEC layer were acquired. Often, it is sufficient to acquire images at a single Z position and quantify the number of migrated cells in the gel at targeted Z position. As an example, images at the 120 μm Z plane were quantified using the Z-slicing feature of IN Cell Analyzer 3000 (GE Healthcare). Images were analysed using the Object Intensity analysis module. The experiments were repeated multiple times, such that each data point—in the Figures—represents mean plus/minus standard deviation of 6 replicate wells, one Z plane of image/well.

Results

Preparation of Collagen Gel

Proper gel formation is essential for quality TEM assay. FIGS. 2 and 3 show that gel quality affects TEM results significantly. The broken gel was prepared by inserting pipette tip through the gel layer, or creating a big air bubble into the gel. As a comparison, control gel at various volumes was dispensed into each well using 12-channel pipette. Air bubbles seem to be a major factor contributing to variation of TEM assay for both neutrophil and PBMC. Broken gel does affect the assay results as compared to the normal control gel, but with less significance. It is noted that small air bubbles were carried into the gel easily by extra force when ejecting gel using multi-channel pipette. We found that a 1,500 rpm spin of the plate for 2 min at 4° C. removes most of the air bubbles. It is also noted that handling the wash process carefully could prevent broken gel from happening.

Gel volume is also critical for the assay set up due to the limitation of the analytical instrument. The IN Cell Analyzer can only focus to a limited Z distance of 200 μm into the gel from the bottom of the plate. Our analysis demonstrates that 40μl gel volume provides a good gel depth for forming a layer at the center of the well, and satisfies the requirement of the assay as well as the instrument. FIGS. 4 and 5 show the effects of gel volume on neutrophil and PBMC TEM, respectively.

HUVEC Culture

HUVEC from CAMBREX was cultured in EGM-2 medium according to the Materials and Methods section above. A proper confluent monolayer of HUVEC culture was grown on the collagen gel. This was confirmed by cadherin-5 immuno-staining. The color image on the right of FIG. 1 shows a confluent endothelial cell monolayer with tight junctions. Cell nuclei are stained by Hoechst (blue). F-Actin is stained by Alexa Fluor™ 488 conjugated phalloidin (green). The red staining reveals a protein, VE-Cadherin, which is expressed at cell boundaries when a tight-junction is formed.

Neutrophil and PBMC Seeding Density

Seeding density of neutrophil and PBMC was titrated to identify the optimum cell number needed for the assay. The neutrophil starting cell density analysis result is shown in FIG. 6. It is shown that a density of 300,000-500,000 cell/well is required to achieve a reasonable signal to noise (S/N) ratio. A similar range was defined for PBMC.

Time Course of Neutrophil and PBMC TEM

Neutrophil TEM happened relatively quickly and reached a significant S/N (IL-1 β stimulated vs. non-stimulated) in about 0.5- 2 hours. FIG. 7 shows the result of a neutrophil TEM time course assay. The migrated cells were quantified at Z: 120 μm above the plate bottom at time points of 0.5, 1, 1.5 and 2 hours, respectively. We also noticed that the neutrophil TEM S/N (with/without IL-1 β stimulation) reached a plateau with an over-night incubation.

PBMC TEM happened relatively slowly, requiring an incubation time of 6-8 hours in general. FIG. 8 shows the result of a PBMC TEM time course assay. The migrated cells were quantified at Z: 120 μm above the plate bottom at time points of 2, 4, 6, and 8 hours, respectively.

IL-8 Soaking Increases Neutrophil TEM

IL-8 is a known strong neutrophil attractor. To demonstrate IL-8's effect on neutrophil TEM, collagen gel with a layer of a confluent HUVEC monolayer was pre-soaked with culture medium containing IL-8 at 200 ng/ml for 4 hours, prior to starting the assay. FIG. 9 shows results of this study. The results indicate that the soaking of IL-8 generates a TEM effect similar to that of IL-1 β activation of HUVEC. IN+/IN−: presence/absence of an MMP-9 inhibitor (see below).

Inhibition of Neutrophil TEM by MMP-9 Inhibitors

Prior to the TEM assay, neutrophil were pre-treated with 1, 10-phenathronoline, a MMP-9 inhibitor (12-1000 μM) for 0.5 hour. The inhibitor was continuously present throughout the TEM assay. Inhibition of neutrophil TEM is demonstrated in FIG. 9 and 10. Each data point in FIG. 10 represents mean plus/minus SD of 6 replicate wells, one image/well at Z: 60 μm. Another MMP-9 inhibitor, doxycyclin was also tested and showed to inhibit TEM (data not shown).

A 3-Dimensional Reconstituted TEM Image

A visualized 3-D cell image of leukocyte TEM in a well of a 96-well plate is illustrated in FIG. 11. Z-series of confocal image sections were generated using IN Cell Analyzer 3000. In total, images from 21 slices from 0-200 μm through the gel layer at 10 μm per section were acquired. The 3-D image was built using image analysis program AutoDeblur&AutoVisulize 9.3 (AutoQuant Imaging). Note that FIG. 11 shows only a small portion of a well, with a field view of about 0.75mm². This 3-D image demonstrates leukocyte TEM in the gel layer, migrating downwards to the gel containing chemoattractant.

Large Scale Study of Neutrophil TEM

A scatter plot of neutrophil TEM, presented as number of migrated cells at Z: 120 μm, with or without HUVEC activation, is shown in FIG. 12. The tight-scatter distribution indicates that variation within the treatments is very small and the difference between two treatments gives significant signal window which qualifies the assay for high throughput applications.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. A composition of matter for detecting migration of cells, comprising:
(a) a solid layer comprising collagen gel or synthetic matrix gel;
(b) a first cellular layer in contact with said solid layer and comprising a first cell type; and
(c) a second migrating cell type seeded on top of said first cellular layer.

2. The composition of claim 1, wherein said migration of cells is transendothelial migration (TEM).

3. The composition of claim 1, wherein said solid layer further comprises a fluorogenic compound.

4. The composition of claim 1, wherein said solid layer further comprises a chemoattractant.

5. The composition of claim 4, wherein said chemoattractant is added into said collagen gel prior to the addition of said first cellular layer.

6. The composition of claim 4, wherein said chemoattractant is released by said first cell type of said first cellular layer after cytokine stimulation.

7. The composition of claim 1, wherein said first cellular layer is confluent.

8. The composition of claim 1, wherein said first cell type is human umbilical vein endothelial cells (HUVEC).

9. The composition of claim 1, wherein said first cell type is stimulated by cytokine.

10. The composition of claim 1, wherein said second cell type is selected from the group consisting of monocyte, neutrophil, lymphocyte, natural killer cell, tumor cells or spermatozoa.

11. The composition of claim 1, wherein said second cell type is neutrophil or peripheral blood mononuclear cells (PBMC).

12. The composition of claim 1, wherein said second cell type is dye labelled.

13. The composition of claim 1, in a 96 wells plate format.

14. The composition of claim 13, wherein said first cell type is a confluent layer of HUVEC, and wherein said second cell type is a neutrophil or a PBMC.

15. The composition of claim 1 in a 384 wells plate format.

16. The composition of claim 13 further comprising an automated fluorescence microscope for image acquisition.

17. The composition of claim 16, whereas said automated fluorescence microscope includes a confocal microscope and software for automated image acquisition and simultaneous on-line analysis.

18. A method for preparing a composition of matter for the detection of cell migration, comprising the steps of:
(a) depositing and solidifying collagen gel in a vessel to form a solid layer comprising collagen gel;
(b) placing cells of a first cell type on said solid layer and incubating said first cell type to form a confluent cellular layer in contact with said solid layer; and
(c) seeding cells of a second cell type on top of said first cellular layer.

19. A method of detecting cell migration, comprising the steps of:
(a) incubating said composition of claim 1; and
(b) detecting migrated cells at a first position of said solid layer of the composition.

20. A method for identifying a mediator of cell migration comprising:
(a) incorporating a candidate mediator of cell migration into the composition of claim 1;
(b) incubating said composition; and
(c) measuring cell migration in the presence of said candidate mediator, wherein a difference in response relative to a composition lacking said candidate mediator identifies a mediator of cell migration.

21. The composition of claim 1, wherein said solid layer further comprises gelatine.

22. The method of claim 18, wherein said depositing step further includes mixing a gelatine solution with said collagen gel.

23. The composition of claim 15 further comprising an automated fluorescence microscope for image acquisition.

24. The composition of claim 23, whereas said automated fluorescence microscope includes a confocal microscope and software for automated image acquisition and simultaneous on-line analysis.
